(19) United States
(12) Patent Application Publication
(10) Pub. No.: US 2022/0351520 A1
(43) Pub. Date: Nov. 3, 2022

(54) APPARATUS, SYSTEM AND METHOD FOR REAL TIME HAND HYGIENE MONITORING

(71) Applicant: PRESHA HEALTH TECH PRIVATE LIMITED, Nagpur (IN)

(72) Inventors: PARTH KOTHARI, Mumbai (IN); Shobha Ram Patle, Mumbai (IN); Rochin Patle, West Malling (GB); Vidhi Patle, West Malling (GB)

(21) Appl. No.: 17/762,976

(22) PCT Filed: May 5, 2020
(86) PCT No.: PCT/GB2020/051099

§ 371 Date: Mar. 23, 2022

(30) Foreign Application Priority Data
Sep. 23, 2019 (GB) 1913672.0

(51) Int. Cl. G06V 20/52; G06V 10/14; G06T 7/70; G06V 10/74; G06V 40/20

(52) U.S. Cl. CPC G06V 20/52; G06V 10/14; G06T 7/70; G06V 10/74; G06V 40/28; G06T 2207/30196; G06T 2207/30232

(57) ABSTRACT

The invention provides an apparatus, system and method for monitoring a user's hand hygiene procedure in real time for comparison with an approved hand hygiene procedure to monitor compliance. The apparatus has an infra-red camera (11), a light source (12) and linked software processing unit (9) connected to a wireless transmitting antenna (10). Another compartment (7) houses disinfectant/soap dispenser refills (6). The memory stores data relating to an approved hand hygiene procedure, and the camera supplies image data relating to the user's hand hygiene procedure to the processor. The processor compares image data supplied by the camera with data stored in the memory relating to the approved hand hygiene procedure to monitor compliance with the approved hand hygiene procedure and stores the results of the comparison in the memory.

### TECHNICAL FIELD

The present invention relates to an apparatus, system and method for monitoring hand hygiene and in particular to an apparatus, system and method for monitoring a user's hand hygiene procedure in real time for comparison with an approved hand hygiene procedure to monitor compliance.

### BACKGROUND

Effective implementation of hand hygiene practice by health care staff in the care of patients is a very important aspect in the health care industry. Failure of effective hand hygiene has been found to be an important factor in cross infections, including antibiotics resistance, in health care institutes, resulting in a significant financial and social burden across the world.

The World Health Organisation (WHO) has developed very comprehensive strategic and resource guidelines for health care institutes to help in sustaining effective hand hygiene practice by health care staff. Health care institutes across the world also have to compulsorily provide their hand hygiene compliance data for WHO surveys and other regulatory schemes. A significant amount of time and resources are therefore spent by institutes to ensure optimum training for staff.

The WHO has published comprehensive resources and tool kits on the following website: https://www.who.int/infection-prevention/tools/hand-hygiene/en/

To summarise the guidelines in the context of the present invention, health care staff are regularly trained and monitored to follow an effective hand hygiene procedure by using either an alcohol-based handrub or a soap- and water-based handwash procedure. The WHO has published guidance for when hand hygiene should be performed in a “My 5 Moments for Hand Hygiene” approach, illustrated in FIG. 1 and here: https://wwwwho.int/infection-prevention/campaigns/clean-hands/5moments/en/

An example of an alcohol-based handrub or a soap- and water-based hand wash procedure is shown in FIG. 2 and here: https://medical.unon.org/flu-vaccines

The objectives of hand hygiene surveillance are to assess the following:
1. Use of the required amount of disinfecting handrub or handwash;
2. Contact of the handrub/handwash for the required duration; and
3. Use of the correct technique to ensure that all parts of the hands are cleaned with the handrub/handwash, thus ensuring an effective technique every time.

The key steps of the correct technique are as follows:
Step 2: Rub hands palm to palm.
Step 3: Rub the back of your left hand with your right palm with interlaced fingers. Repeat with the other hand.
Step 4: Rub your palms together with fingers interlaced.
Step 5: Rub the backs of your fingers against your palms with fingers interlocked.
Step 6: Clasp your left thumb with your right hand and rub in rotation. Repeat with your left hand and right thumb.
Step 7: Rub the tips of your fingers in the other palm in a circular motion, going backwards and forwards. Repeat with the other hand.

### SUMMARY OF THE INVENTION

In accordance with a first aspect, the invention provides an apparatus for monitoring a user's hand hygiene procedure in real time, comprising a camera, a processor and a memory, wherein the memory stores data relating to an approved hand hygiene procedure, and wherein the camera supplies image data relating to the user's hand hygiene procedure to the processor, the processor being configured to compare image data supplied by the camera with data stored in the memory relating to the approved hand hygiene procedure to monitor compliance with the approved hand hygiene procedure and store the results of the comparison in the memory.

In a preferred embodiment, the processor is configured to analyse the image data to determine the extent to which a cleaning agent (e.g. disinfectant, soap, alcohol-based rub) is applied to the user's hands. The cleaning agent may be detectable more reliably by using illumination, e.g. by means of light or infra-red light. Therefore in this embodiment, or in any other applicable embodiment, the apparatus preferably further includes a light source to illuminate the user's hands during the hand hygiene procedure.

The emitted light may be in the visible or non-visible spectrum. The light source may be modified by means of a filter or other suitable means to emit light of a specific wavelength or from a specific range of wavelengths, e.g. ultra violet or infra-red. Preferably the light source is an infra-red source and the camera is an infra-red camera. The cleaning agent may be modified to include a tracer to improve its visibility at certain light wavelengths.

The image data may comprise real time tracking data relating to the position and/or movement of the user's hands, fingers and/or thumbs during the hand hygiene procedure and the stored data comprises tracking data relating to the position and/or movement of hands, fingers and/or thumbs during the approved hand hygiene procedure. The memory may further store augmented or virtual reality software for use by the processor during comparison of the tracking data.

The image data may alternatively or in addition comprise real time images of the user's hands, fingers and/or thumbs during the hand hygiene procedure and the stored data includes images of hands, fingers and/or thumbs during the approved hand hygiene procedure. The user's images may for example be superimposed on approved images to check compliance.

The apparatus preferably further includes a timer for timing the user's hand hygiene procedure, the timer supplying timing data to the processor for comparison with timing data stored in the memory relating to an approved hand hygiene procedure.

The apparatus preferably further includes a controller connected to the processor which, on receipt of a signal from the controller, controls the dispensing of a cleaning agent (e.g. on receipt of user ID information) or the supply of water for rinsing (e.g. on satisfactory application of cleaning agent).

In accordance with a second aspect, the invention provides a system for monitoring a user's hand hygiene, comprising the apparatus discussed above and a user-specific interface device.

In preferred embodiments, the user-specific interface device comprises an ID card, which may include a photo, barcode, or RFID tag.

Preferably, the apparatus includes a transmitter and the user-specific interface device includes a receiver, the apparatus transmitting data to the receiver relating to the user's hand hygiene. The data may be transmitted via any suitable system, such as the internet, Wi-Fi or Bluetooth. The transmitted data may comprise or include a reminder signal or alarm.

In accordance with a third aspect, the invention provides a method for monitoring a user's hand hygiene procedure in real time with an apparatus comprising a camera, a processor and a memory, the memory storing data relating to an approved hand hygiene procedure, the method comprising the steps of supplying image data of the user's hand hygiene procedure from the camera to the processor, comparing image data supplied by the camera with data stored in the memory relating to the approved hand hygiene procedure to monitor compliance with the approved hand hygiene procedure, and storing the results of the comparison in the memory.

There are some hand hygiene monitoring systems available in the market for retrospective data analysis, but none are designed to objectively assess and encourage good hand hygiene practice in real time. Our invention is designed to help health care institutes to improve their hand hygiene practice amongst the staff and to improve the quality of data to comply with WHO and other regulatory surveillance schemes.

Our invention analyses the key steps involved in the guidelines to achieve effective hand hygiene:
1. Application of disinfectant (hand wash or alcohol rub). Our invention incorporates a camera (infra-red thermal camera) with a specific wavelength filter in the infra-red spectrum. This enables visualisation of the flow of disinfectant handwash or alcohol handrub based on the infra-red light emitting properties (also known as emissivity) of the various ingredients of handrub, especially the main ingredient which is ethanol. The alternative approach will be to add a tracer fluid to commonly used hand wash soaps, which can be picked up by the applied specific wavelength filter on the camera. This camera will capture images of soap/handrub covered hands and help in identifying effective soap application. The live video feed of flow of the hand wash soap will be timed and relayed to the processor.
2. Real time three-dimensional hand and finger movement tracking. This is achieved by linking the camera to a software program to digitally track and analyse the movement of each finger/thumb and hands. Suitable software programs may be found in augmented and virtual reality systems.
3. Real time monitoring. Timer software will be installed in the camera, to ensure that each hand hygiene action follows the required duration as mentioned in guidelines.
4. Real time verification of hand hygiene procedure with the guidelines. The images from the camera are relayed to the processing unit, where they are superimposed to verify the handrub/soap application method and hand/finger gestures, by comparison with saved images from official control guidelines and specifications for an effective hand hygiene procedure.
5. Compliance monitoring system. The system includes a data storage system to function as a compliance log, for each member of staff. The equipment also has blue tooth and web connectivity so that reminder alarms can be sent to staff members for regular hand hygiene action as per the regulatory policies. Staff can be provided with a peripheral signal receiver device with an individual bar code such as an ID card for this purpose.

In the case of a handwash procedure employing a conventional basin and tap, a tap controller or an electronic flow valve may be provided which is controlled by a signal from the processor. When the processor validates a compliant hand hygiene procedure by matching image files of the user's procedure with stored control image files in real time, only then an activating electronic signal is provided to the electronic flow valve, which will allow the tap water to restart to help rinse off the soap/disinfectant. The tap will not restart the water flow until such signal is received from the image processor, and thus the objective of the invention is to implement the hand hygiene protocol.

An integral part of the invention is the electronic circuit board/processor connecting the different components:
1. bar code scanner (for the staff) and button for the visitor;
2. disinfectant hand wash or hand rub dispenser;
3. image sensors/lenses with infra red filter and image processing/storage unit and wireless connections; and
4. electronic flow valve placed/installed around the plumbing pipe of the wash basin, controlling the tap water flow.

An individual hand hygiene procedure starts with user identification (staff or visitor) e.g.

by means of a bar code scanner for staff or a button for visitors. This is connected to the data storage unit for hand hygiene compliance records.

The user identification leads to the timed activation of water flow for an initial rising of hands, after which the water flow stops and the camera lens/image sensor is activated to monitor the application of soap or disinfectant. The soap may be dispensed at this point either automatically by the system, or manually by the user. Following real time validation of the procedure, the water flow is reactivated to allow rinsing. For staff at least, the procedure and the results may then be stored for compliance monitoring purposes.

In the case of a handrub procedure, typically water is not required, so there will not be a tap controller. The dispensing of the handrub will be activated (or may be manually dispensed) after user identification (staff or visitor) e.g. by means of a bar code scanner for staff or a button for visitors. The camera lens/image sensor will then be activated to monitor the correct application of handrub. Following real time validation of the procedure, the procedure and the results may then be stored for compliance monitoring purposes, at least for staff and optionally for visitors.

This invention is the only system which is designed for real time monitoring of a hand hygiene procedure for implementation of WHO guidelines for hand hygiene techniques. Many studies have shown that the prevalence of satisfactory hand hygiene compliance in healthcare staff tapers down to a very low percentage in the weeks following training or direct observation of their hand hygiene technique. This invention is the first of its kind to help staff follow the protocol in real time as it compels staff to follow the standard operating procedures as advised by the WHO, the failure to implement such procedures having very big burden in the health care economy.

### BRIEF DESCRIPTION OF THE DRAWINGS

An embodiment of the invention will now be described by way of example only and with reference to the accompanying drawings, in which:

FIG. 1 shows the “My 5 Moments for Hand Hygiene” approach published by the WHO;

FIG. 2 shows an example of an alcohol-based handrub or a soap- and water-based hand wash procedure;

FIG. 3 shows a first embodiment of the invention installed on a wash basin;

FIG. 4 shows a second embodiment of the invention installed at an alcohol handrub station;

FIG. 5 shows a peripheral user ID card;

FIG. 6 shows a process flow chart for the invention;

FIG. 7 shows an overview of a hand wash console in accordance with a third embodiment of the invention;

FIG. 8 shows the interior of the hand wash console of FIG. 7;

FIG. 9 shows various components of the hand wash console of FIGS. 7 and 8;

FIG. 10 shows the hand wash console of FIGS. 7-9 in use;

FIG. 11 shows an overview of a hand rub console in accordance with a fourth embodiment of the invention;

FIG. 12 shows the interior of the hand rub console of FIG. 11; and

FIGS. 13 to 17 show a design of hand wash console in accordance with a fifth embodiment of the invention.

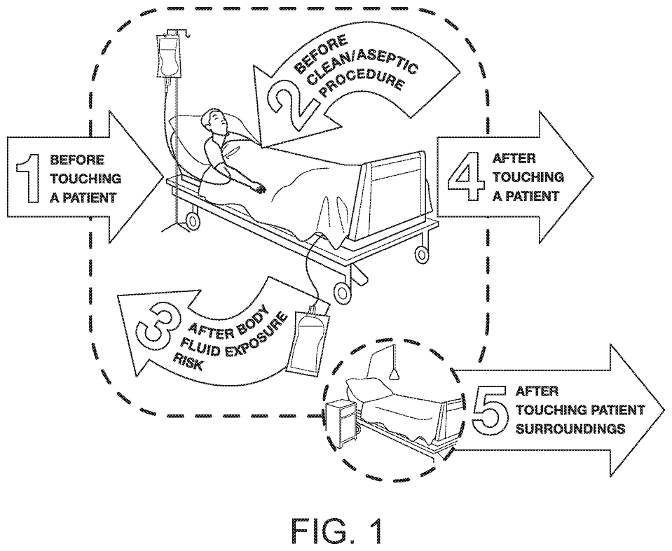

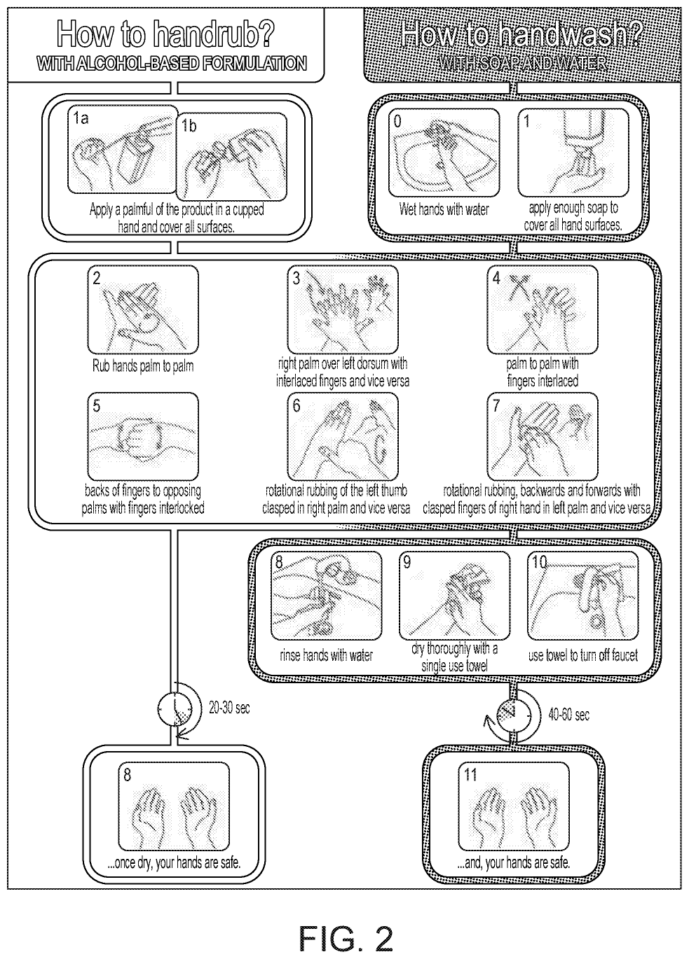

### DETAILED DESCRIPTION OF PREFERRED EMBODIMENTS

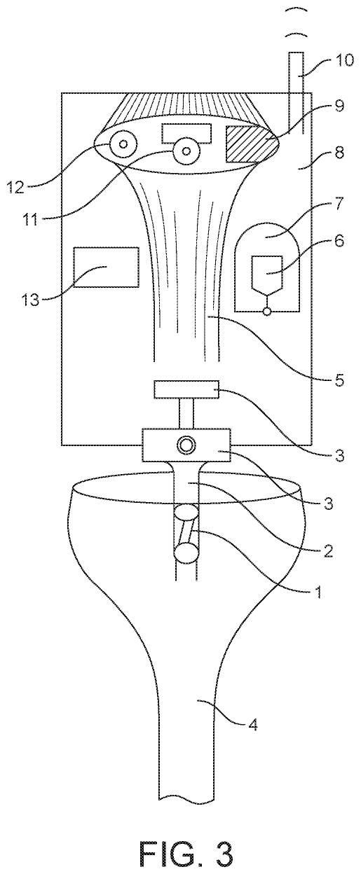

FIG. 3 shows a first embodiment of the invention installed on a wash basin 4. The embodiment consists of a flat plastic board 8 which could be fixed on the wall or on a mobile trolley. The board has compartments to house different components of the system interconnected by electronic circuits and processing units housed in a column 5. The top part has a compartment, which houses infra-red camera 11, a light source 12 and linked software processing unit 9 connected to a wireless transmitting antenna 10. Another compartment 7 houses disinfectant/soap dispenser refills 6.

A barcode and infra-red sensor 13 is provided on one side of the board 8 which is activated by the user card and is linked to data storage and the transmission connection.

The lower part of the equipment comprises a water flow tap 3 and a plumbing pipe 2 with an electronic water flow switch 1 to control the flow of water as per the electronic signals.

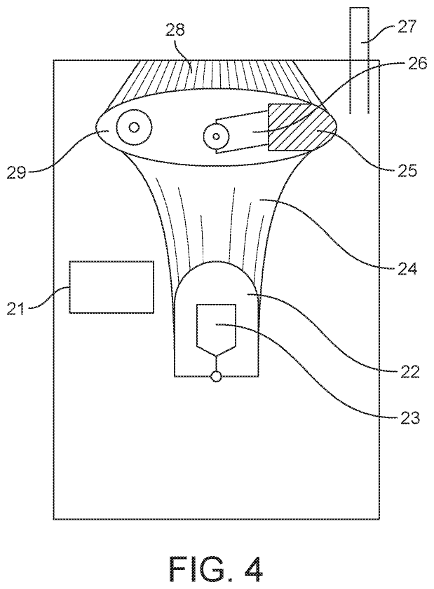

FIG. 4 shows a second embodiment of the invention installed at an alcohol handrub station. The modified smaller version consists of similar plastic board with a compartment 28 to house light source 29, infra-red camera 26 with linked image processing unit 25 connected to a wireless signal transmitting antenna 27. The electronic connections and processing unit are housed in plastic column 24. The compartment 22 is for housing the alcohol rub dispensing unit 23. The board is activated by the barcode/infra-red sensor 21.

The two embodiments of the invention are designed for handwash and handrub hand hygiene routines respectively. In a busy hospital, multiple such units may be installed in different locations. The wireless connectivity and data transfer from each equipment could be through local area wireless network and server computers. The units would have appropriate electronic/software installations to enable the formation of a network of equipment communicating with each other and the staff/users through the ID card.

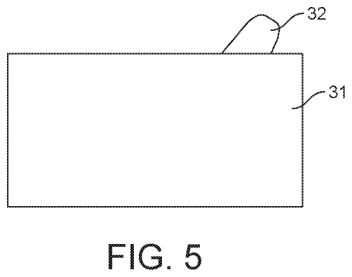

The peripheral signal alert receiver/user ID card 31 given to registered staff for hygiene compliance monitoring is shown in FIG. 5. The card has a light or vibrating system 32.

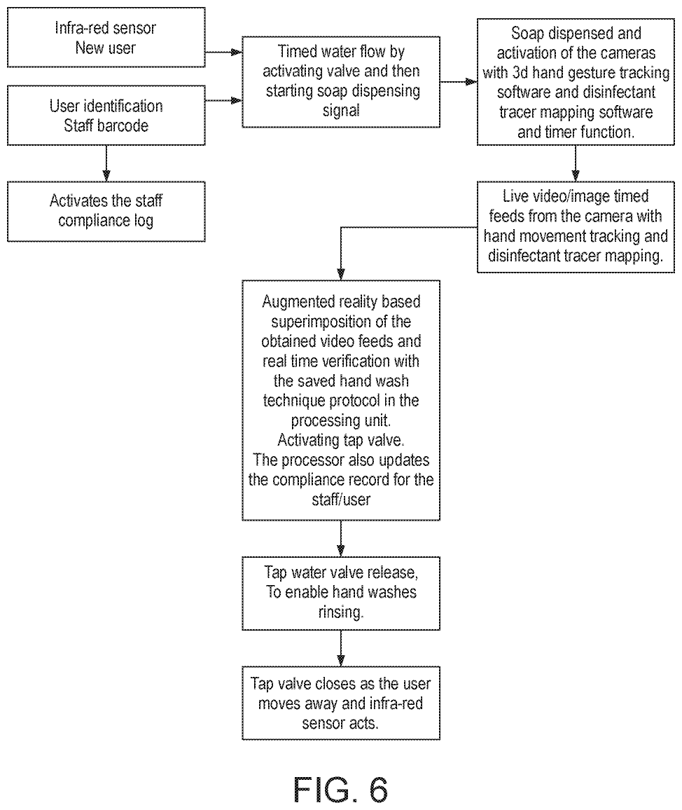

FIG. 6 shows a process flow chart for the invention.

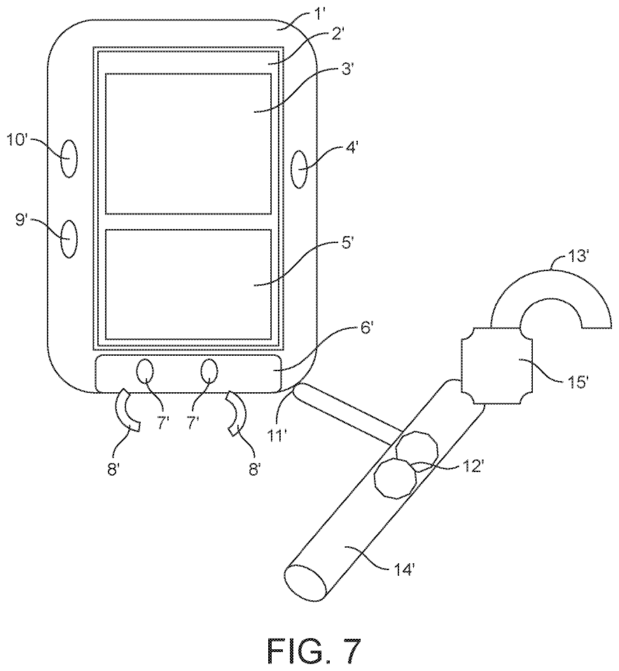

FIGS. 7-10 shows a hand wash console in accordance with a third embodiment of the invention. With reference to FIG. 7, the embodiment consists of a hard plastic board (1′), which would be fixed on the wall over the wash basin area. The console has switches to start the function, which may be an infra-red sensor, a barcode scanner or a radio frequency identification tag reader (4′,9′,10′). There is a box cabinet (2′) covering most of the plastic board, which is approx. 12×8×6 inches in dimension. The bottom of the box will be transparent, made up of an infra-red wavelength selecting filter (6′) to accommodate camera lenses (7′) and having openings for nozzles (8′). The front wall of the box cabinet will have a mirror (3′) and a LED screen for displaying messages (5′). The console is connected (via connector 11′) to the electronic flow valve (12′) installed in the plumbing pipe (14′), to control the flow of water through connector (15′) and wash basin faucet (tap) (13′).

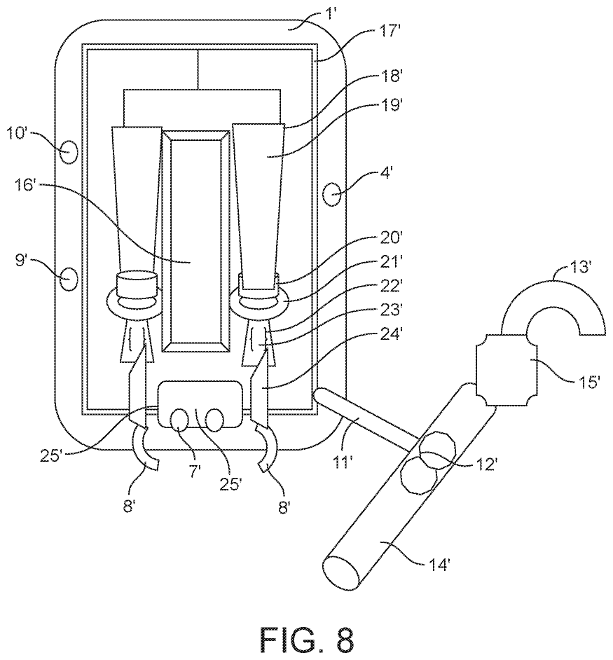

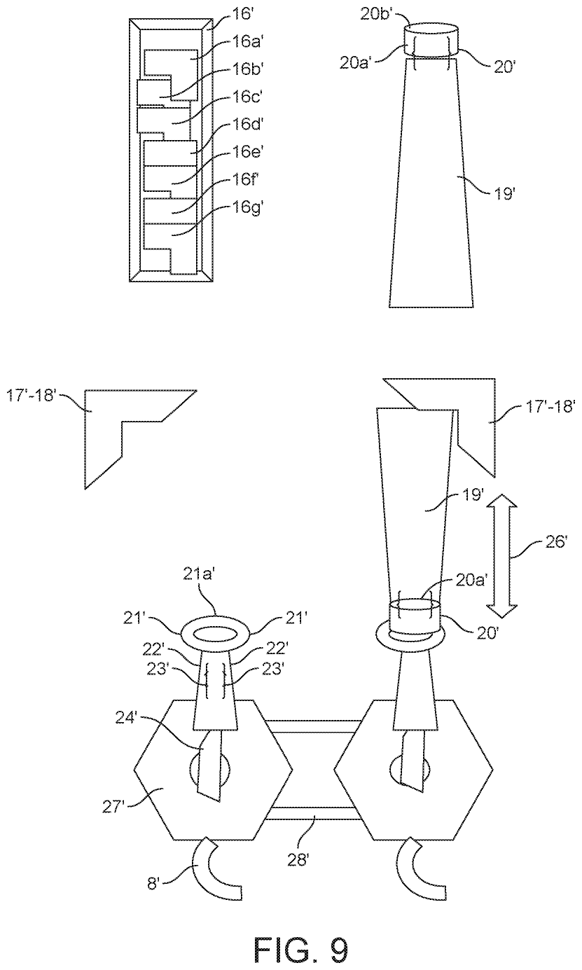

With reference to FIGS. 8 and 9, the box cabinet is shown open to reveal the components. The inside of the back wall of the box cabinet (2′) has two equal size elongated grooves to accommodate two hand wash dispensing bottles (19′) made up of plastic and located in an upside down position (FIGS. 8, 9). The bottles are positioned between the spring loaded brackets (17′, 18′) on top of the box cabinet supporting the base of the bottles and by a specially designed cap rim sliding sleeve connector (21′, 22′) made up of plastic or metal. The cap rim sliding sleeve connector slides up and down (26′, FIG. 9) on the sharp straw-nozzle system, made up of plastic or metal (8′, 24′), this system is fixed and is not mobile on the bottom floor of the box cabinet, the sharp straw is inside continuing into the nozzle outside as seen in FIG. 9.

The hand wash bottle fixes on the cap-rim through ridges on the bottle cap (20′), this cap is made up of flexible material with a ridge on top surface (20b′). The bottle moves up and down along with the cap rim sliding sleeve on top of the straw-nozzle system over a spring (22′), when compressed by the brackets (17′, 18′) to dispense the wash solution as the sharp straw enters the opening on the bottle cap (20a′) into the bottle, resulting in flow of solution through the nozzles (FIG. 9).

There is a camera body (25′) and lens (7′) installed between the two nozzles, to capture the images of two hands placed under the nozzle to receive the dispensed wash solution. The whole mechanism is automated and works in a programmed sequence controlled by the processor box (16′) placed between the two bottles and above the camera unit. FIG. 9 shows the components in this box 16′: (a-g) these are series of integrated circuit boards for central processing unit, Ram, Timer function, power supply, internet of things wireless connectivity, user interface, interconnectivity of components, software kernel, data storage.

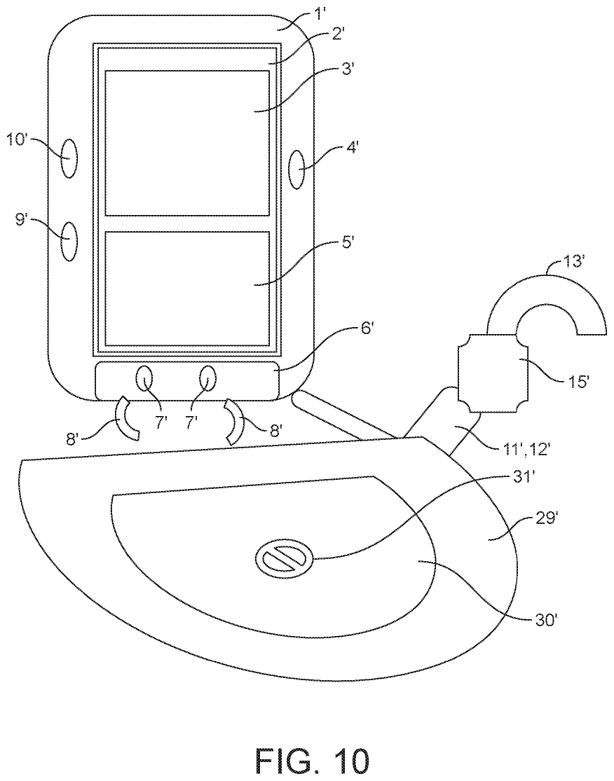

FIG. 10 shows the hand wash console installed over a wash basin (29′, 30′, 31′). The console is placed in such a way that the dispensing nozzles are positioned over the wash basin for drips to drain, and thus the tap faucet is placed on one side of the wash basin.

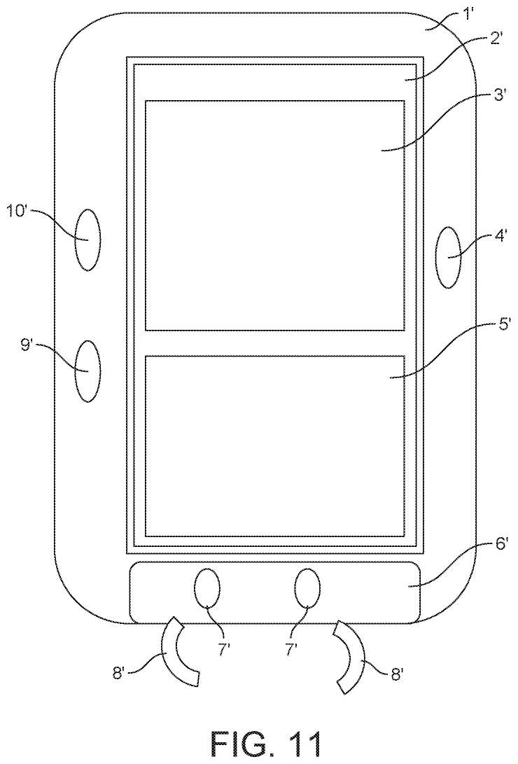

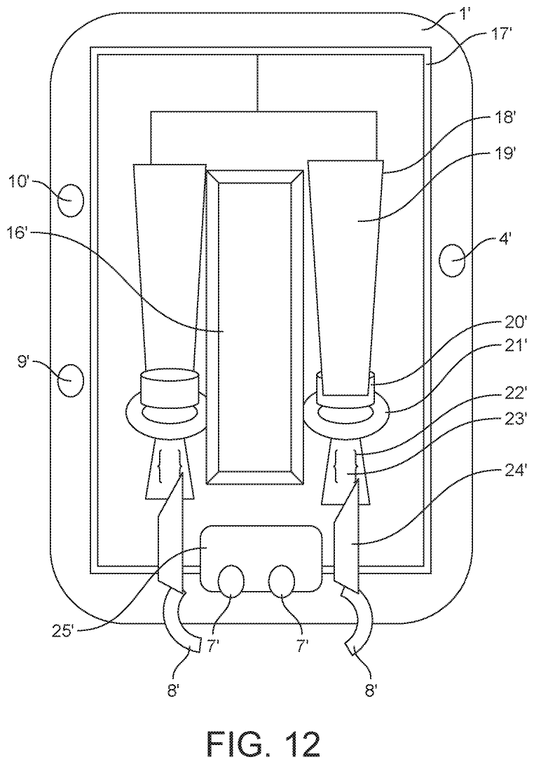

FIGS. 11 and 12 show a hand rub console in accordance with a fourth embodiment of the invention with similar components and function to the third embodiment, except this application does not have an extension to the electronic flow valve and faucet. The same reference numbers are employed for the components as for the third embodiment.

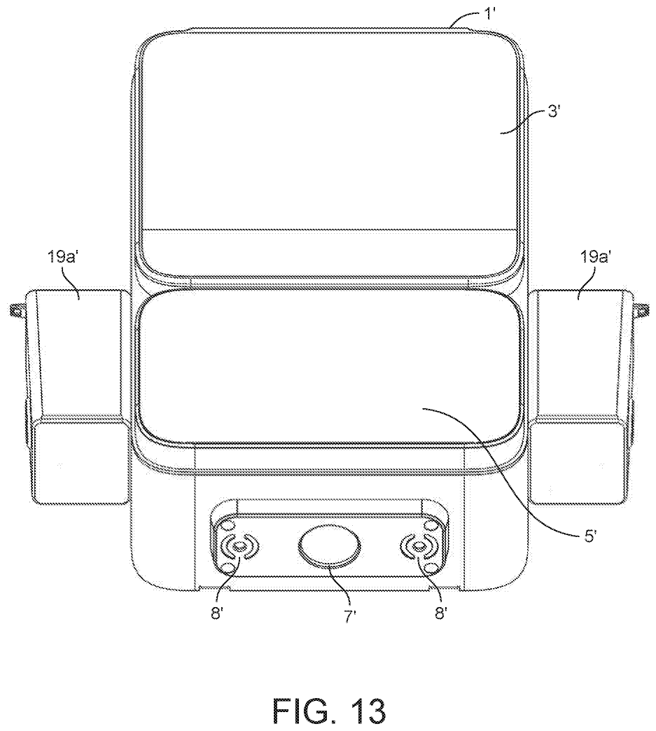

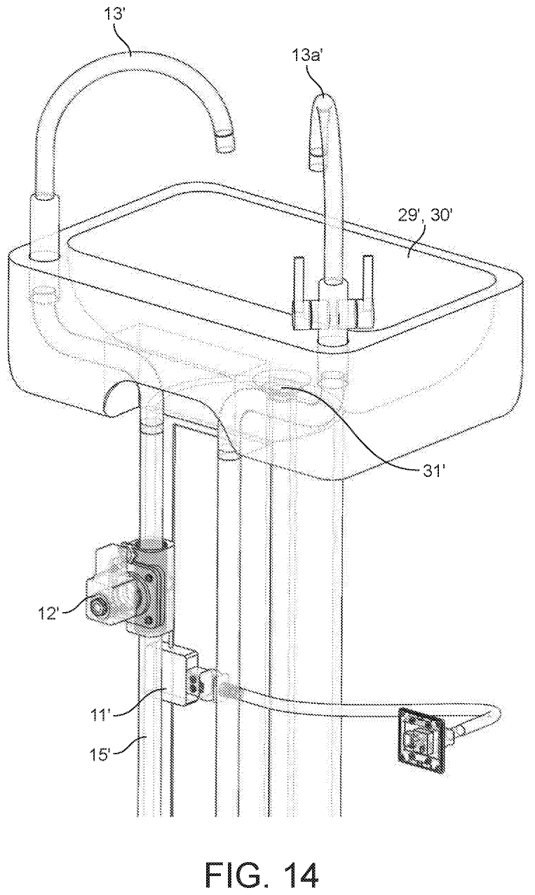

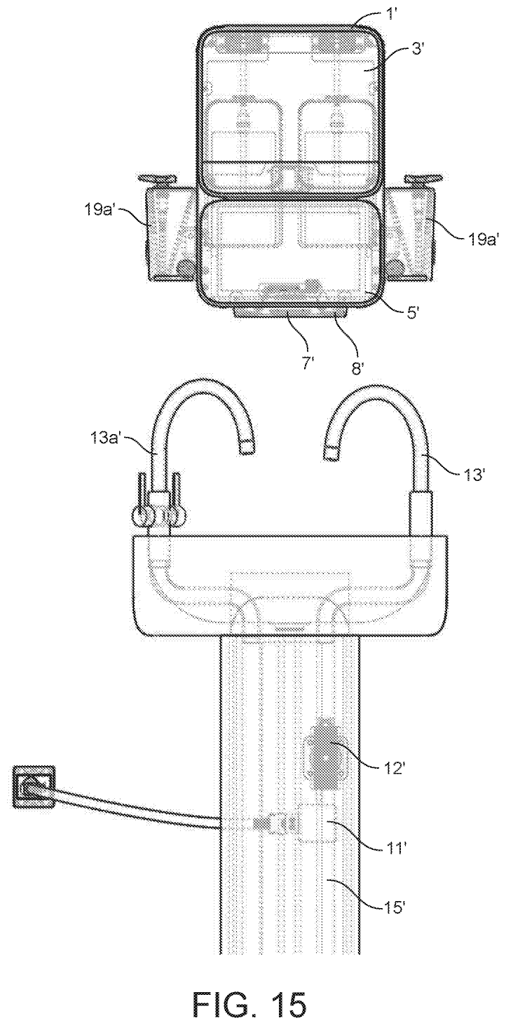

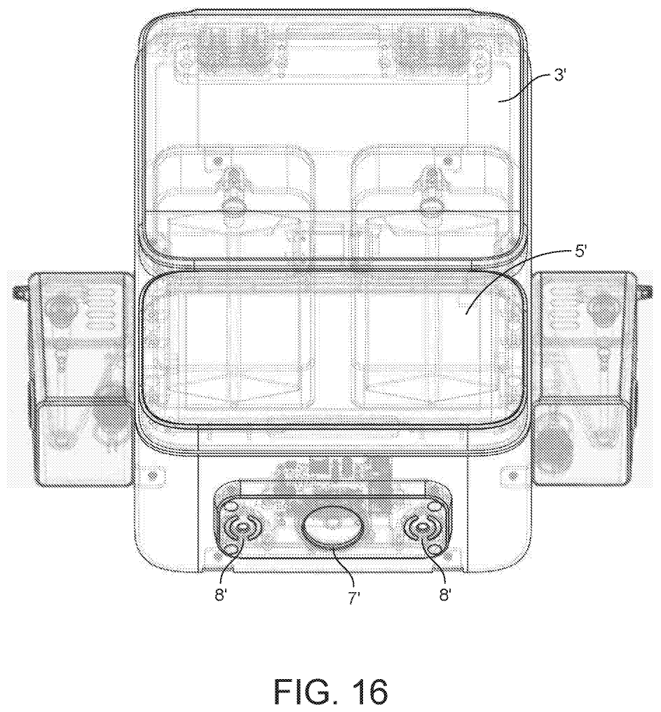

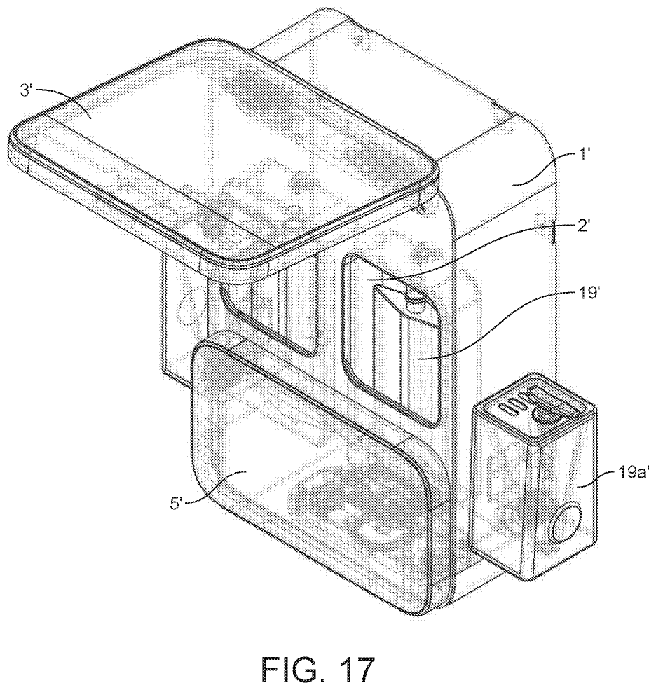

FIGS. 13-17 show a design of hand wash console in accordance with a fifth embodiment of the invention, which is similar in many respects to the third embodiment discussed above. Similar reference numbers are employed for the components.

Our invention with its features and design is the first of its kind to:
1. Capture real time images of hand hygiene procedures with disinfectant/soap or with alcohol by tracking hand and finger positions to identify the required gestures (steps 2-7) as suggested and numbered above to verify a compliant hand hygiene process;
2. Monitor in real time the duration of a hand hygiene procedure;
3. Validate in real time validation the hand hygiene procedure in comparison with guideline images;
4. Capture the application of disinfectant/soap or alcohol rub on the hand in real time through infra-red imaging based on the infra-red emission properties of disinfectant/soap or alcohol; and
5. Capture and process the image files for processing, storage and transmission.

Our invention is the first of its kind to involve a dual hand soap dispensing system. This invention is the first of its kind to integrate a soap dispensing system with a water flow control mechanism to enable a WHO hand hygiene compliant hand wash or hand rub procedure.

In summary, the invention provides an augmented reality enabled real time compliance implementing hand hygiene monitoring system.

Further technical features or benefits of the invention include:
1. Dispensing hand wash or a hand hygiene product on two hands at the same time;
2. Capturing images, saving and processing the image files in the software processing unit;
3. Detecting and tracking the hand and finger movements in real time;
4. Integrating the hand hygiene product dispensing with the electronic flow valve and tap/faucet mounted on the wash basin; and
5. The functions of detecting peripheral units, such as sensors and codes embedded in staff id cards, and wireless communication and data transfer.

Business Method

A further aspect of the invention lies in a business method, utilising the apparatus of any of the embodiments of the invention.

The apparatus may be supplied as a unit together with any of the following additional components of an overall system: washbasin, plumbing, electrical wiring, IT connectivity (for example to a local or cloud-based network), peripherals including user ID cards and/or sensors.

The apparatus may be termed a “smart hand hygiene unit” or “smart wash basin”. The apparatus may perform some or all of the following functions to deliver tangible benefits to its customers:
1. The apparatus will dispense hand hygiene product and objectively analyse, through its software algorithm of machine learning of real time image analysis and validation of hand hygiene techniques for the most effective hand hygiene episode. This will generate detailed data relating to the compliance of hand hygiene techniques used by the users for audit purposes.
2. The system may provide a feature of setting a timer or user location based reminder for the user to carry out a hand hygiene episode through its networked peripheral sensors for e.g. tracking the user location such as a toilet or other potentially-contaminated area, reminding the user and logging the subsequent hand hygiene episode for the user (or failure to comply). The system will generate a report for the customer.
3. The system may also remind the user/customer of hand hygiene product levels and place online orders for automatic replacement. Trouble shooting may be provided online or via a cloud network.

In commercial terms, a novel business method may involve a subscription-based payment model for usage of the apparatus. Customers may purchase staff cards for their number of employees and make regular monthly or yearly payments as per any software service subscription. The service may include a managed service contract element. This novel business method may be referred to as “Hand Hygiene as a Software Service (HhaSS)”.

In some examples, the service provider and user/institutional client would have a business service contract. The service would provide the most effective hand hygiene events/episode for every user of the equipment by real time automated verification and validation of the user's hand hygiene technique. The service may provide the user of the service with a compliance report of hand hygiene technique for every staff of the institutional user/customer/client. The business may also maintain the supply of the consumables including hand hygiene products by automated inventory management. The business may also provide wireless communication to staff to remind them to take appropriate hand hygiene action when required.

The details mentioned above involve a new invention and a new method of business, both of which do not exist in current market.

This smart dispenser integrated wash basin will ensure that most effective hand hygiene technique is practised/implemented for every user each time by real time automated image analysis and controlled activation of the wash basin faucet to complete a hand hygiene episode.

1. Apparatus for monitoring a user's hand hygiene procedure in real time, comprising a camera, a processor and a memory, wherein the memory stores data relating to an approved hand hygiene procedure, and wherein the camera supplies image data relating to the user's hand hygiene procedure to the processor,
the processor being configured to:
compare image data supplied by the camera with data stored in the memory relating to the approved hand hygiene procedure to monitor compliance with the approved hand hygiene procedure, and
store the results of the comparison in the memory.

2. The apparatus of claim 1, wherein the processor is configured to analyze the image data to determine the extent to which a cleaning agent is applied to the user's hands.

3. The apparatus of claim 1, further comprising a light source to illuminate the user's hands during the hand hygiene procedure.

4. The apparatus of claim 3, wherein the light source is an infra-red source and the camera is an infra-red camera.

5. The apparatus of claim 1, wherein the image data comprises real time tracking data relating to the position and/or movement of the user's hands, fingers and/or thumbs during the hand hygiene procedure and wherein the stored data comprises tracking data relating to the position and/or movement of hands, fingers and/or thumbs during the approved hand hygiene procedure.

6. The apparatus of claim 5, wherein the memory further stores augmented or virtual reality software for use by the processor during comparison of the tracking data.

7. The apparatus of claim 1, wherein the image data comprises real time images of the user's hands, fingers and/or thumbs during the hand hygiene procedure and wherein the stored data comprises images of hands, fingers and/or thumbs during the approved hand hygiene procedure.

8. The apparatus of claim 1, wherein the apparatus further includes a timer for timing the user's hand hygiene procedure, the timer supplying timing data to the processor for comparison with timing data stored in the memory relating to an approved hand hygiene procedure.

9. The apparatus of claim 1, further comprising a controller connected to the processor which, on receipt of a signal from the processor, controls the dispensing of a cleaning agent or the supply of water for the hand hygiene procedure.

10. A system for monitoring a user's hand hygiene procedure, comprising:
a user-specific interface device;
a processor;
a memory to store data relating to an approved hand hygiene procedure; and
a camera to supply image data relating to the user's hand hygiene procedure to the processor, the processor being configured to:
compare image data supplied by the camera with data stored in the memory relating to the approved hand hygiene procedure to monitor compliance with the approved hand hygiene procedure, and
store the results of the comparison in the memory.

11. The system of claim 10, wherein the user-specific interface device comprises an ID card.

12. The system of claim 11, wherein the ID card includes a photo, barcode, or RFID tag.

13. The system of claim 10, wherein the apparatus includes a transmitter and the user-specific interface device includes a receiver, the apparatus transmitting data to the receiver relating to the user's hand hygiene.

14. The system of claim 13, wherein data is transmitted via the internet, Wi-Fi or Bluetooth.

15. A method for monitoring a user's hand hygiene procedure in real time with an apparatus comprising a camera, a processor and a memory, the memory storing data relating to an approved hand hygiene procedure, the method comprising the steps of:
supplying image data of the user's hand hygiene procedure from the camera to the processor,
comparing image data supplied by the camera with data stored in the memory relating to the approved hand hygiene procedure to monitor compliance with the approved hand hygiene procedure, and
storing the results of the comparison in the memory.
